(19) United States
(12) Patent Application Publication
(10) Pub. No.: US 2026/0256573 A1
(43) Pub. Date: Sep. 3, 2026

(54) POLYMERIC VALVULAR STRUCTURE AND METHOD OF PREPARATION THEREOF

(71) Applicant: MERIL LIFE SCIENCES PVT LTD, Vapi (IN)

(72) Inventors: PRAMOD KUMAR MINOCHA, Vapi (IN); DEVESHKUMAR MAHENDRALAL KOTHWALA, Surat (IN); AMIRHAMZAH MAHMADIQBAL SHAIKH, Valsad (IN); HAROONMOHAMMAD RASIDMOHAMMAD KHALIFA, Valsad (IN)

(73) Assignee: MERIL LIFE SCIENCES PVT LTD, Vapi, GJ (IN)

(21) Appl. No.: 19/158,036

(22) PCT Filed: Feb. 16, 2024
(86) PCT No.: PCT/IN2024/050167

§ 371 Date: Aug. 19, 2025

(30) Foreign Application Priority Data
Feb. 20, 2023 (IN) 202321011507

(51) Int. Cl. A61F 2/24; A61L 27/18; A61L 27/34

(52) U.S. Cl. CPC A61F 2/2415; A61L 27/18; A61L 27/34; A61L 2430/20

(57) ABSTRACT

The present invention discloses a method to prepare a polymeric film for making at least one of a valvular structure (103) and one or more sealing means (102) of a prosthetic heart valve. The method commences by pre-processing a polymer for a pre-defined time to reduce residual moisture content of the polymer. Thereafter, the pre-processed polymer is dissolved in at least one solvent yielding a polymer solution having a pre-defined concentration. The polymer solution is poured into an enclosed surface having a pre-defined area. The polymer solution is subjected to a drying technique at a first pre-defined temperature(s) and a first pre-defined pressure(s) for a first pre-defined time period(s) to yield a polymeric film having a thickness ranging from 150 microns to 220 microns. The polymeric film is washed and subjected to the drying technique at a second pre-defined temperature(s) for a second pre-defined time period(s).

### FIELD OF INVENTION

The present invention relates to a method of preparing a valvular structure. More specifically, the present invention relates to a method of preparing a polymeric valvular structure for a transcatheter heart valve.

### BACKGROUND OF INVENTION

A native heart valve is made of a plurality of leaflets. The leaflets rhythmically coapt with each other to facilitate unidirectional flow of blood within the chambers of the heart. Over time, the native heart valve may deteriorate and cause life threatening conditions in an affected person. One of the major suspects for the said deterioration of the native heart valve is gradual calcium depositions (also known as calcification) over the surface of the native leaflets thereby causing damage to the native heart valve. The damaged native heart valve fails to maintain the physiological hemodynamics of a healthy heart and cause valvular insufficiency (also known as valvular regurgitation). In order to treat the damaged native heart valve, the native heart valve is replaced with a prosthetic heart valve.

Previously, the prosthetic heart valves could be implanted only via open-heart surgery requiring extracorporeal blood circulation. Given the fragility of heart patients, open heart surgery with extracorporeal blood circulation amounted to fatal consequences for most patients. In fact, many patients could not even qualify for such a sophisticated surgery thereby being denied treatment for their heart conditions.

To remedy the situation, minimally invasive procedures in the form of transcatheter aortic valve implantation (TAVI) or transcatheter heart valve replacement (TAVR) was introduced. TAVI is a percutaneous process in which the prosthetic transcatheter heart valve (THV) is implanted via a small puncture through the skin of a patient.

Generally, a THV includes an artificial valvular structure made of three leaflets supported inside an expandable frame. The leaflets of the artificial valvular structure mimic the function of healthy native leaflets thereby maintaining healthy blood flow. The leaflets of the artificial valvular structure are usually made of animal tissues (for example, bovine tissue, porcine tissue, etc.).

However, valvular structure made of an animal tissue may compromise on the hemocompatibility of the valve's material thereby providing inferior hydrodynamic performance. Further, owing to the differences in tissue degeneration and tissue lifespan of the two different species i.e., native human tissue and animal tissue of the artificial valvular structure, the risk of heart failure, stroke, or even death of patient receiving the said artificial valvular structure increases significantly. Furthermore, implantation of foreign tissue from another species always carries a risk of disease transfer from the animal to the human.

Hence, prior using animal tissue for making leaflets, it is mandatory to treat the tissue using fixative agents (usually to prevent immune response against the foreign tissue post implantation). Most common fixative agents used to fix animal tissues are aldehydes like glutaraldehyde. However, post treatment of the tissue with the fixative agent, the tissue includes an abundance of free aldehydes. These free aldehydes act as a potential calcium binding site. Thus, after implantation of the THV having the animal tissue-based leaflets, the free aldehydes induce rapid calcification of the artificial valvular structure in a short span of time thereby reducing the lifespan of the THV.

Further, the animal tissue-based leaflets include a rough surface that is prone to calcification, thrombosis and degeneration thereby limiting its durability.

In order to overcome the aforementioned disadvantages and limitations of animal tissue-based leaflets, THVs having polymeric leaflets are being explored as an alternative. Although polymeric leaflets perform better than the tissue leaflets, there still remain various challenges to be addressed that are associated with polymeric leaflets. For instance, owing to the currently employed methods by which the polymeric leaflets are made, THVs having polymeric leaflets are susceptible to high fatigue stress and degeneration of leaflets thereby resulting in complications such as poor hydrodynamic performance, mineralization, thrombosis and significant decrease in the durability of the THV.

As an example, the patent publication number US20220039946A1 discloses preparation of a polymeric valvular structure by electrospinning method. However, the resultant valvular structure develops a porous surface which attracts blood particles and/or calcium particles thereby resulting in thrombosis and/or calcification of the polymeric leaflets.

Furthermore, the patent publication numbers US20070027535A1 and U.S. Pat. No. 10,266,657B2 teaches preparation of polymeric leaflets by solvent casting method using Dimethylacetamide (DMAc) as the solvent. Due to high boiling point of the DMAc solvent, the time required to evaporate the solvent is very long. Further, the resultant polymeric film obtained for making the polymeric leaflets, would be sticky, semi-solid and/or unstable. In other words, the polymeric film obtained would not be fit for making polymeric leaflets that could be implanted within the body.

Thus, there arises a need to devise a method for preparing a polymeric valvular structure that can overcome the issues related to conventional leaflets.

### SUMMARY OF INVENTION

The present invention relates to a method to prepare a polymeric film for making at least one of a valvular structure and one or more sealing means of a prosthetic heart valve. The method commences by pre-processing a polymer for a pre-defined time to reduce residual moisture content of the polymer to less than 0.02% by weight of the polymer. Thereafter, the pre-processed polymer is dissolved in at least one solvent yielding a polymeric solution having a pre-defined concentration. The solvent having water content less than or equal to 0.005% (w/v). The polymeric solution is poured into an enclosed surface having a pre-defined area. The polymeric solution is subjected to a drying technique at a first pre-defined temperature(s) and a first pre-defined pressure(s) for a first pre-defined time period(s) to yield a polymeric film having a thickness ranging from 150 microns to 220 microns. The polymeric film is washed and subjected to the drying technique at a second pre-defined temperature(s) for a second pre-defined time period(s). The present invention further relates to a prosthetic heart valve having a valvular structure and/or one or more sealing means made of the polymeric film.

The foregoing features and other features as well as the advantages of the invention will become more apparent from the following detailed description, which proceeds with reference to the accompanying figures.

### BRIEF DESCRIPTION OF ACCOMPANYING DRAWINGS

The summary above, as well as the following detailed description of illustrative embodiments, is better understood when read in conjunction with the apportioned drawings. For the purpose of illustrating the present disclosure, exemplary constructions of the disclosure are shown in the drawings. However, the disclosure is not limited to specific methods and instrumentalities disclosed herein. Moreover, those in the art will understand that the drawings are not to scale.

FIG. 1 depicts an exemplary transcatheter heart valve 100 in accordance with an embodiment of the present invention.

FIG. 2 depicts a method 200 for preparing a polymeric film for a valvular structure 103 in accordance with an embodiment of the present invention.

FIGS. 3 and 4 depict the valvular structure 103 made from the polymeric film in accordance with an embodiment of the present invention.

FIG. 5 depicts a method 300 for assembling the transcatheter heart valve 100 in accordance with an embodiment of the present invention.

### DETAILED DESCRIPTION OF DRAWINGS

Prior to describing the invention in detail, definitions of certain words or phrases used throughout this patent document will be defined: the terms “include” and “comprise”, as well as derivatives thereof, mean inclusion without limitation; the term “or” is inclusive, meaning and/or; the phrases “coupled with” and “associated therewith”, as well as derivatives thereof, may mean to include, be included within, interconnect with, contain, be contained within, connect to or with, couple to or with, be communicable with, cooperate with, interleave, juxtapose, be proximate to, be bound to or with, have a property of, or the like; Definitions of certain words and phrases are provided throughout this patent document, and those of ordinary skill in the art will understand that such definitions apply in many, if not most, instances to prior as well as future uses of such defined words and phrases.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Although the operations of exemplary embodiments of the disclosed method may be described in a particular, sequential order for convenient presentation, it should be understood that the disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed herein. Moreover, for the sake of simplicity, the attached figures may not show the various ways in which the disclosed system, method, and apparatus can be used in combination with other systems, methods, and apparatuses.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments. These features and advantages of the embodiments will become more fully apparent from the following description and apportioned claims, or may be learned by the practice of embodiments as set forth hereinafter.

In accordance with the present disclosure, a method of preparing a valvular structure is disclosed. The valvular structure in the present invention corresponds to an assembly of a plurality of leaflets to be used within a prosthetic heart valve, for example, a transcatheter heart valve (THV). The resultant THV may be used in transcatheter aortic valve implantation (TAVI) procedures or the like. Although the present invention is described with examples of transcatheter heart valves, other kinds of prosthetic valves such as surgical heart valves, venous valves, etc. are within the scope of the teachings of the present invention.

The valvular structure of the present invention is made of a polymeric material, such as a polymeric film. Owing to the polymeric film, the valvular structure of the present invention provides relatively better resistance towards calcification, fatigue stress, degeneration leading to superior hemodynamic performance.

The polymeric film for the valvular structure of the present invention is made by a solvent casting technique. Owing to the preparation of the polymeric film for the valvular structure by the solvent casting technique, the resultant valvular structure includes uniform thickness, maximum optical purity and low haziness thereby leading to better hydrodynamic performance and improved life span of the valvular structure.

As an overview, the method of the present invention includes pre-processing of a polymer. The polymer, as selected, plays an important role in proper functioning of the THV. Accordingly, the polymer selected in the present invention includes a biostable and biocompatible polymer that provides sufficient flexibility to the valvular structure thereby allowing smooth and rhythmic coaptation of the leaflets during diastolic and systolic cycles of the heart.

After the polymer is pre-processed, a polymeric film is prepared by a solvent casting technique. The polymeric film, as obtained by solvent casting technique, is then cut in a pre-defined shape and coated to form the valvular structure. The valvular structure formed may then be attached to a frame of the THV for subsequent use.

The valvular structure prepared by the method of the present invention has a nearly smooth surface(s) hence is less susceptible to fatigue stress and provides better hydrodynamic performance. Further due to the nearly smooth surface(s) of the valvular structure, the polymeric valvular structure is resistant to calcification and degeneration thereby rendering the valvular structure durable.

In an exemplary embodiment, the valvular structure derived from the method of the present invention is in the form of a single continuous structure, i.e., the valvular structure includes a plurality of integrally formed leaflets rather than separate leaflets. The single continuous structure of the valvular structure makes it structurally stable, i.e., the valvular structure of the present invention is more resistant towards wear and tear compared to valvular structures formed by stitching separate leaflets to each other. Given the lack of stitching between adjacent leaflets of the valvular structure of the present invention, the chances of paravalvular blood leakage through stitched joints are drastically reduced leading to the increase in lifespan of the THV after implantation.

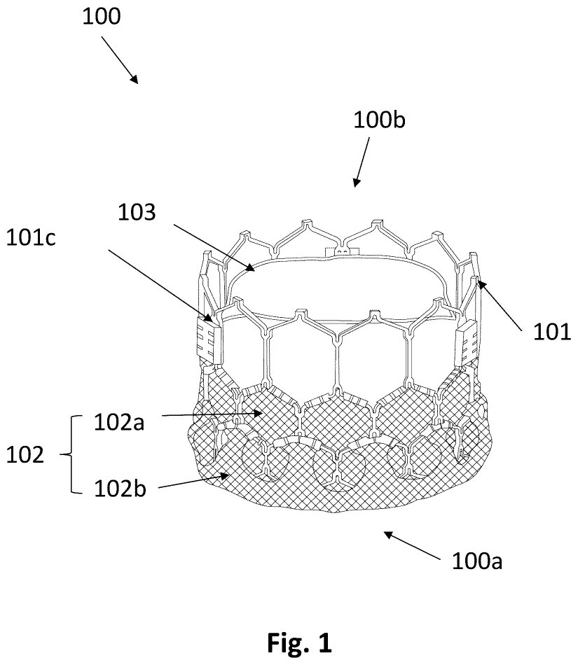

Now referring to figures, FIG. 1 depicts an exemplary embodiment of a transcatheter heart valve 100 (or THV 100). The THV 100 is a device that is implanted inside a patient's body to replace a diseased native heart valve by the functionality of a healthy native valve. The THV 100 is implanted at a target site (not shown) using a delivery catheter (not shown). The THV 100 is mounted over the delivery catheter in a radially compressed state and thereafter, radially expanded once the THV 100 is positioned at the target site. In an exemplary embodiment, the target site includes a native aortic valve of the heart.

The THV 100 may belong to one of the two categories, namely, balloon expandable or self-expandable. In case the THV 100 is a balloon expandable, once the THV 100 is positioned at the target site, a balloon of the delivery catheter is expanded to force the THV 100 to open (or radially expand) by plastic deformation. Thereafter, the balloon is deflated and the delivery catheter is withdrawn leaving the THV 100 fixed against the native heart valve and/or the arterial wall (not shown).

On the contrary, a self-expandable THV 100 is made of a ‘shape memory metal’. The self-expandable THV 100, in its radially compressed state, is held within a sheath of the delivery system during its delivery. Once the self-expandable THV 100 is positioned at the target site, the sheath is withdrawn to reveal the self-expandable THV 100 such that the self-expandable THV 100 radially expands on its own.

The THV 100 may include a plurality of components including but not limited to a frame 101, one or more sealing means 102 and a valvular structure 103. At least one of the plurality of components of the THV 100 may be made from a polymeric film (described below). In other words, at least one of the frame 101, the valvular structure 103 and the sealing means 102 may be made from the polymeric film of the present invention. In an exemplary embodiment, the valvular structure 103 of the THV 100 is made from a polymeric film. In alternate embodiment, the sealing means 102 of the THV 100 is made from the polymeric film. In yet another embodiment, the sealing means 102 and the valvular structure 103 are made from the polymeric film. Further, the THV 100 includes an inflow end 100a and an outflow end 100b. The THV 100 may be balloon expandable or self-expandable. In an exemplary embodiment, the THV 100 is balloon expandable.

The frame 101 corresponds to an anchoring structure or a structural framework of the THV 100. The frame 101 is a tubular structure made of a bioresorbable and/or bio-compatible material(s) selected from metals or polymers. For example, the frame 101 may be made of a metallic material including but not limited to binary Nickel-Titanium alloy (nitinol), ternary Copper-Zinc-Aluminum alloy, Copper-Aluminum-Nickel alloy (or any other copper-based alloys), Cobalt Chromium alloy, etc. Alternatively, the frame 101 may be made of a polymeric material including but not limited to Poly(L-lactide) (PLLA), Poly(D, L-lactide-co-glycolide) (PLGA), Poly(ε-caprolactone) (PCL), Poly(D, L-lactide-co-glycolide) (PLGA), etc. In an exemplary embodiment, the frame 101 is made of Cobalt Chromium alloy.

The sealing means 102 may be in the form of an inner skirt 102a and/or outer skirt 102b, that covers the frame 101 at least partially. The inner skirt 102a may be disposed at an inner surface of the frame 101 and the outer skirt 102b may be disposed at an outer surface of the frame 101. The sealing means 102 prevent paravalvular blood leakage post implantation of the THV 100. In an exemplary embodiment, the sealing means 102 are made of a polymeric film. In an alternate embodiment, the sealing means 102 are made of Polyethylene terephthalate (PET) fabric.

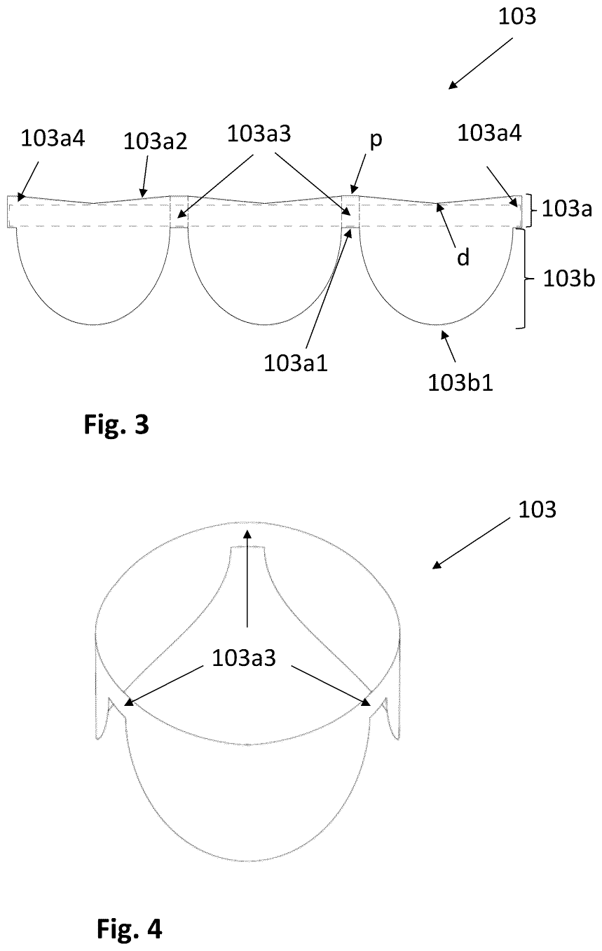

The valvular structure 103 is supported within the frame 101. In an exemplary embodiment, the valvular structure 103 is stitched to the inside of the frame 101. The valvular structure 103 may include a plurality of leaflets that mimic the function of healthy native leaflets. The number of leaflets that form the valvular structure 103 may depend upon the damaged native valve i.e., whether the damaged native valve is bicuspid/tricuspid, etc. In an exemplary embodiment, the valvular structure 103 that is intended to replace a tricuspid damaged native valve includes three leaflets. In an exemplary embodiment, as shown in FIGS. 3 and 4, the leaflets of the valvular structure 103 forms a single continuous structure. In an alternate embodiment, a valvular structure (not shown) is formed by stitching discrete leaflets to each other. The single continuous structure of the valvular structure 103 eliminates wear and tear of the leaflets compared to when discrete leaflets are stitched together.

The valvular structure 103 as per the teachings of the present invention is made of a polymeric film that performs better compared to valvular structure being made of animal tissues. For example, the valvular structure 103 made of polymeric film provides relatively better resistance towards calcification, fatigue stress, and degeneration, etc. leading to superior hemodynamic performance.

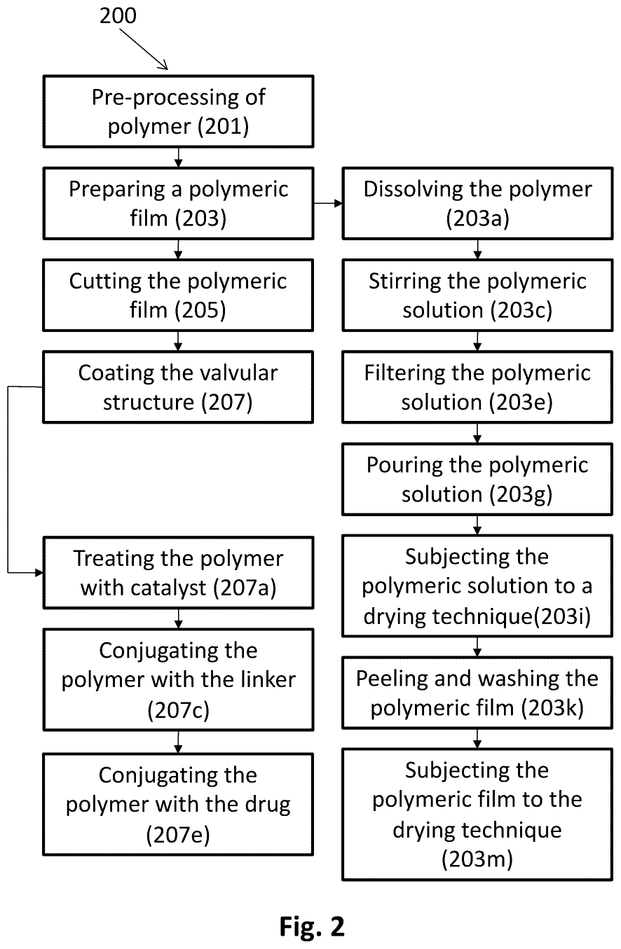

FIG. 2 depicts a method 200 for preparing the polymeric film for making the valvular structure 103.

The method 200 may be executed to prepare either the single continuous structure of the valvular structure 103 or to prepare the valvular structure having discrete leaflets. The method 200 may be used to prepare the sealing means 102 as well.

The valvular structure 103 may be made of a biostable and/or biocompatible polymer. The biostable and biocompatible polymer may be selected from a group of polyurethane or block co-polymers thereof including but not limited to polyurethane, poly ether urethane, polycarbonate urethane, etc. The use of biostable and/or biocompatible polymers provide sufficient flexibility to the valvular structure 103 thereby allowing easy rhythmic movement of the leaflets of the valvular structure 103 during diastolic and systolic cycles of the heart. In an exemplary embodiment, the polymer used to form the valvular structure 103 is polycarbonate urethane block co-polymer. Polycarbonate urethane block co-polymer provides resistance to calcification, thrombogenesis and degradation thereby increasing the operating life of the THV 100 to more than 15 years.

The method 200 commences at step 201 in which the polymer is subjected to pre-processing. The polymer may be selected from a group of polyurethane or block co-polymers thereof including but not limited to polyurethane, poly ether urethane, polycarbonate urethane, etc. The pre-processing of the polymer includes drying the polymer for a pre-defined time to remove moisture (or dehydrate). The pre-defined time may be optimized for minimal to none residual moisture content in the polymer. The pre-defined time may range from 12 hours to 24 hours. The polymer may be in the form of pellets, powder, granules, etc. In an exemplary embodiment, the polymer is in the form of pellets. The polymer can be dried using a pre-defined method including but not limited to a vacuum desiccator, a vacuum oven, a desiccant type de-humidifying dryer, a hot air oven, etc. In an embodiment, the polymer pellets are dried in a vacuum desiccator for 24 hours at a temperature of 37±5° C.

It should be noted that the polymer (in solid and/or un-dissolved state) as available to be used for pre-processing may be hygroscopic in nature, i.e., the polymer may absorb moisture from the surrounding environment. Any residual moisture in the polymer leads to formation of bubbles and haziness during subsequent steps of the method 200. The formation of bubbles and haziness may directly affect the mechanical properties (such as decrease in elongation and tensile strength) of the valvular structure 103 thereby reducing the durability of the valvular structure 103. Thus, pre-processing the polymer by drying the polymer helps to reduce any residual moisture thereby avoiding formation of bubbles and haziness in subsequent steps of the method 200 (and the valvular structure 103 obtained at the end). In an exemplary embodiment, the residual moisture content of the polymer at the end of step 201 is reduced to less than 0.02% by weight of the polymer.

At step 203, a polymeric film is formed from the pre-processed polymer obtained at step 201. In an exemplary embodiment, the polymeric film is formed by a solvent casting technique. The preparation of the polymeric film by solvent casting technique ensures uniform thickness, maximum optical purity and low haziness of the polymeric film thereby leading to better hydrodynamic performance and improved life span of the valvular structure 103 (and the THV 100).

The solvent casting technique to obtain the polymeric film is executed in a controlled environment such that the mechanical properties (such as elongation, tensile strength, etc.) of the valvular structure 103 (and/or the polymer) is preserved. The controlled environment corresponds to an enclosed space with a pre-defined humidity and temperature. The humidity may range from 25% to 50% and the temperature may range from 18° C. to 28° C. In an exemplary embodiment, the humidity and temperature of the controlled environment is maintained at 33±1% and 22±1° C. respectively. The aforesaid humidity and temperature of the controlled environment enables preparation of clear and transparent polymeric film without any haziness.

At step 203a of the solvent casting technique, the (pre-processed) polymer may be dissolved in a suitable solvent, for example a polar organic solvent at a pre-defined concentration to yield a polymer solution. The pre-defined concentration ranging from 3% to 11% (w/v). The polar organic solvent may be at least one of dimethyl acetamide (DMAc), tetrahydrofuran (THF), dimethyl sulfoxide (DMSO), and the like. Similar to the dehydrated polymer, anhydrous solvents are preferred for preparation of the polymeric film. In an exemplary embodiment, the solvent having water content less than or equal to 0.005% (w/v) is preferred for dissolving the polymer.

Further, the polymer may be dissolved in a single solvent or in a blend of two or more solvents in a pre-defined ratio in order to preserve the mechanical properties (such as elongation, tensile strength, etc.) of the polymer in the obtained polymeric film (and the valvular structure 103). For example, the polymer may be dissolved in a blend of two solvents having different boiling points. In an exemplary embodiment, a blend of DMAc (having high boiling point) and THF (having low boiling point) is used as the solvent to dissolve the polymer. The aforesaid blend of two solvents facilitates evaporation of the solvents at an even pace (not very slow and no very fast) during subsequent steps (described below) thereby creating a stable and homogenous polymeric film with good morphological and mechanical properties.

In an exemplary embodiment, 6% (w/v) of the polymer is dissolved in a blend of anhydrous DMAc and THF. The anhydrous DMAc and THE may be blended in a predefined ratio of 90:10, 80:20, 70:30, 60:40, 50:50, 40:60, 30:70, 20:80, or 10:90. In an exemplary embodiment, the anhydrous DMAc and THE is blended in the ratio of 70:30. The aforesaid ratio helps in the formation of the polymeric film with desired thickness having excellent morphological (for example, transparency, surface features, homogeneity, etc.) and mechanical properties (for example, force, stress, elongation, young modulus, etc.). For example, 6% (w/v) polymer dissolved in a blend of anhydrous DMAc and THF, blended in a ratio of 70:30, results in a polymeric film with a thickness of 180 microns. Additionally, the thickness of the polymeric film depends upon a volume of the polymer solution poured and an area over which the polymer solution is poured (described below).

As an exemplary embodiment, following Table 1 shows the relationship between concentration of polyurethane copolymer and the thickness of the polymeric film that may be obtained by the solvent casting technique (the following table assumes that the volume of the polymer solution and the area over which the polymer solution is poured is kept constant with variations in the polymer concentration only):

TABLE 1
Polyurethane copolymer concentration and thickness

| Polymer concentration | Thickness of polymeric film |
|---|---|
| (w/v) | (±10 microns) |
| 5% | 140 |
| 5.5% | 155 |
| 6% | 170 |
| 6.5% | 185 |
| 7% | 200 |
| 7.5% | 215 |
| 8% | 230 |
| 8.5% | 245 |
| 9% | 260 |
| 9.5% | 275 |
| 10% | 300 |

At step 203c, after the polymer is dissolved in the solvent, the obtained polymer solution may be stirred to make it homogenous. The polymer solution is stirred using a predefined technique. The predefined technique may include, without limitation stirring motors, magnetic stirrers, overhead geared stirrer, etc. In an exemplary embodiment, the polymer solution is stirred using an overhead geared stirrer. The stirring of the polymer solution is done at the room temperature at a predefined rotational speed for a pre-defined time. In an exemplary embodiment, the solution is stirred at 1500 rotations per minute (rpm) for 7-8 hours. Stirring the polymer solution at the aforesaid rotational speed and pre-defined time eliminates any possibility of formation of clumps or residues and allows the polymer to be completely dissolved in the solvent thereby rendering the polymer solution homogenous.

At step 203e, optionally or additionally, the stirred polymer solution may be filtered through a mesh filter to remove any particulate matter (if present or accidentally introduced).

At step 203g, the polymer solution may be poured over an enclosed surface having a pre-defined area. The enclosed surface corresponds to any bounded flat surface that prevents fluidic run-off. The pre-defined area of the enclosed surface may depend on a size of the prosthetic valve, for example the THV 100. The pre-defined area may range from 80 cm² to 315 cm². In an exemplary embodiment, the polymer solution is poured at a center of a glass petri dish having an area of 254 cm², such that the polymer solution is allowed to spread over an entire area of the glass petri dish.

At step 203i, the aforementioned enclosed surface with the polymer solution is then subjected to a drying technique at a first pre-defined temperature and first pre-defined pressure for a first pre-defined time period(s) to evaporate the solvent(s) from the polymer solution and yield the polymeric film. The first pre-defined temperature may range from 40° C. to 80° C. The first pre-defined pressure may range from 600 mmHg to 700 mmHg and the first pre-defined time period(s) may range from 5 hours to 10 hours. The drying technique includes, without limitation, drying in a vacuum oven, a hot air oven, etc.

In an exemplary embodiment, the glass petri dish containing the polymer solution is placed inside a vacuum oven. Inside the vacuum oven, the enclosed surface containing the polymer solution is gradually heated to evaporate the solvent. Firstly, the polymer solution is maintained at 40° C. at a pressure of 650 mmHg for 3 hours and then the temperature is gradually increased to 70° C. and maintained for 8 hours. The aforesaid gradual heating prevents bubble formation and haziness in the obtained polymeric film. Thus, subjecting the polymer solution to the aforesaid first drying technique yields a clear, stable and homogenous polymeric film with desired and even thickness. The thickness of the resultant polymeric film may range from 150 microns to 220 microns. The aforesaid thickness range of the polymeric film provides better hydrodynamic performance to the valvular structure 103 without any regurgitation. In an exemplary embodiment, the thickness of the polymeric film is 180±10 microns. The valvular structure 103 made from the polymeric film having the aforesaid thickness provides large effective orifice area (EOA) for better hydrodynamic performance and enables low-profile crimping of the THV 100 over the delivery device.

At step 203k, post evaporating the solvent, the polymeric film may be carefully peeled away from the petri dish. In an exemplary embodiment, the polymeric film is peeled away from its edges using forceps. Thereafter, the polymeric film may be washed to remove any solvent residue and/or stickiness. In an exemplary embodiment, the polymeric film is washed twice with distilled water.

At step 203m, the polymeric film is subjected to the drying technique at a second pre-defined temperature for a second pre-defined time period(s). In an exemplary embodiment, the polymeric film is maintained at 37° C. for 24 hours under atmospheric pressure. In an alternate exemplary embodiment, the polymeric film is maintained at 60° C. for 3-4 hours. However, care should be taken not to subject the polymeric film to higher temperatures for long duration as it may melt the polymeric film thereby causing the polymeric film to be sticky and rendering it unfit to make the valvular structure 103. The drying technique at the second pre-defined temperature and time period(s) helps to evaporate any residual moisture from the polymeric film that may be introduced when the polymeric film was washed.

At step 205, the polymeric film obtained from the above step is cut to a pre-defined shape. The polymeric film may be cut using a pre-defined cutting technique including, without limitation, laser cutting, water knife, ultrasonic trimming and the like. In an exemplary embodiment, the polymeric film is cut by laser cutting. The laser cutting process includes feeding the polymeric film into a laser cutting machine followed by directing and impinging a laser beam onto the polymeric film to cut the film into the pre-defined shape.

In an exemplary embodiment, the polymeric film is cut to form the shape of a single continuous structure of the valvular structure 103 as shown in FIG. 3. The valvular structure 103 cut in the aforesaid shape provides smooth washout to the blood thereby minimizing stress, preventing degeneration and resisting thrombosis on the leaflets of the valvular structure 103. Moreover, the single continuous structure of the valvular structure 103 eliminates the need of stitching discrete leaflets thereby reducing wear and tear of the valvular structure 103 and contribute towards longer lifespan of the THV 100 after implantation. Due to absence of any stitched joints between adjacent leaflets of the valvular structure 103, chances of paravalvular blood leakage through the said joints are reduced significantly.

In an exemplary embodiment, the single continuous structure of the valvular structure 103 made of the polymeric film includes three integral leaflets as shown in FIG. 3 (described below in detail). In an alternate embodiment, the valvular structure (not shown) made of the polymeric film includes three discrete leaflets. In other embodiments, the valvular structure 103 includes fewer leaflets than three depending upon the type of native valve the prosthetic valve (for example, the THV 100) is supposed to replace.

Optionally or additionally, at step 207, a pre-defined coating is disposed over the valvular structure 103 as obtained in the step 205. The pre-defined coating of the valvular structure 130 helps to repel any particulate matter present in the blood thereby further minimizing risk of thrombosis and valve failure.

In an exemplary embodiment, the valvular structure 103 is coated only if the receiving patient (person with a diseased native heart valve) is not dependent upon any blood thinners or anti-coagulant agents in their daily life.

In an alternate embodiment, the valvular structure 103 without any coating is prescribed if the receiving patient is dependent on blood thinners or anti coagulating agents in their daily life or patients who have high thrombin time where the blood clotting proteins do not produce thrombin enzyme leading to inhibition of fibrin formation from fibrinogen and ultimately failing to produce fibrin clots.

The pre-defined coating may be coated and/or conjugated over the valvular structure 103 with agents including but not limited to, anti-thrombotics, thrombolytics, antiproliferatives, anti-inflammatories, antimitotic, antimicrobial agents, etc.

In an exemplary embodiment, the pre-defined coating includes anti-thrombotic agents. The anti-thrombotic agents may include, without limitation anti-platelet or anti-coagulants. In an exemplary embodiment, an anti-coagulant agent such as heparin is used to coat the valvular structure 103. Heparin is a glycosaminoglycan which prevents thrombosis over a surface of the valvular structure 103 that actively come in contact with the blood.

In an alternate embodiment, the pre-defined coating is a conjugation of anti-coagulant with polycarbonate urethane (PCU). The said conjugation may be done using a pre-defined coating technique including, without limitation covalent linkage, physical adsorption, ionic bonding and photo-grafting.

In an exemplary embodiment, heparin is conjugated to the polymer by a three-step covalent bonding technique. The first step 207a (depicted below), PCU polymer's surface may be treated with a catalyst such as di-n-butyl tin dilaurate (DBTL) to link the isocyanate functional group i.e., hexamethylene diisocyanate (HDI) to generate HDI linked PCU. In order to prevent the absorption of isocyanate into the PCU polymer's surface, poor solvents such as toluene, xylene, and the like may be used for the first step.

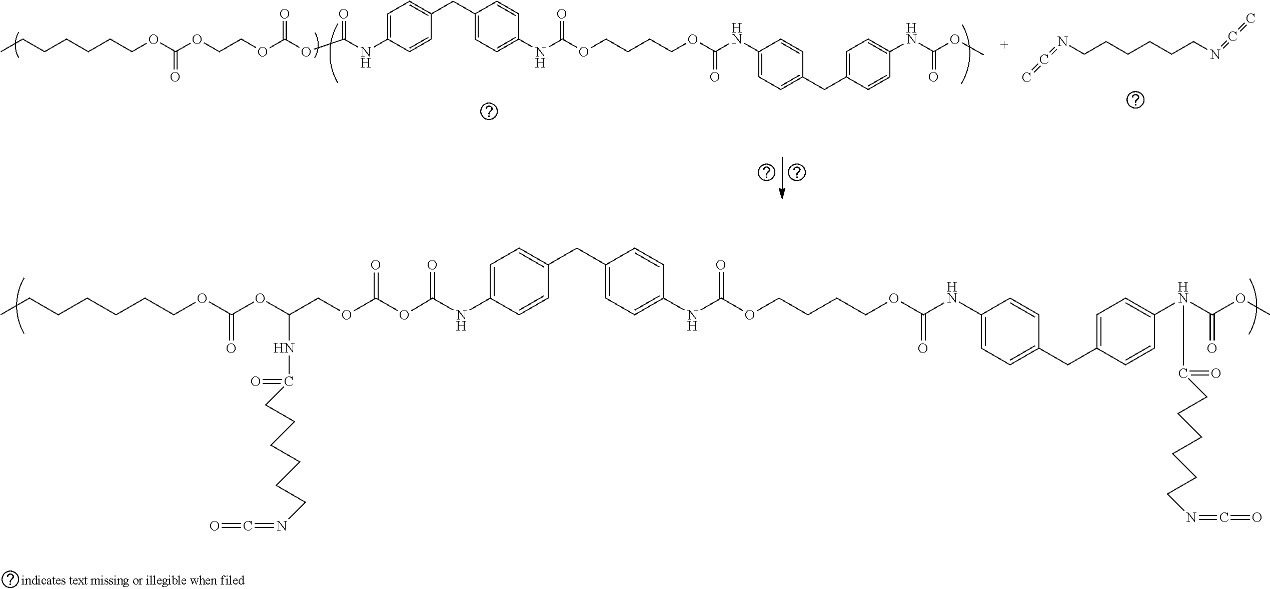

The second step 207c (depicted below) includes conjugating the HDI linked PCU polymer's surface to a, w-diamino polyethylene glycol (APEG) to produce the APEG conjugated PCU polymer's surface. The APEG acts as a spacer (or linker) between the valvular structure 103 and the pre-defined coating (i.e., the heparin) which improves the ability of heparin to form a direct, adherent, and strong bonding with the polymer's surface while decreasing the adsorption of non-specific proteins.

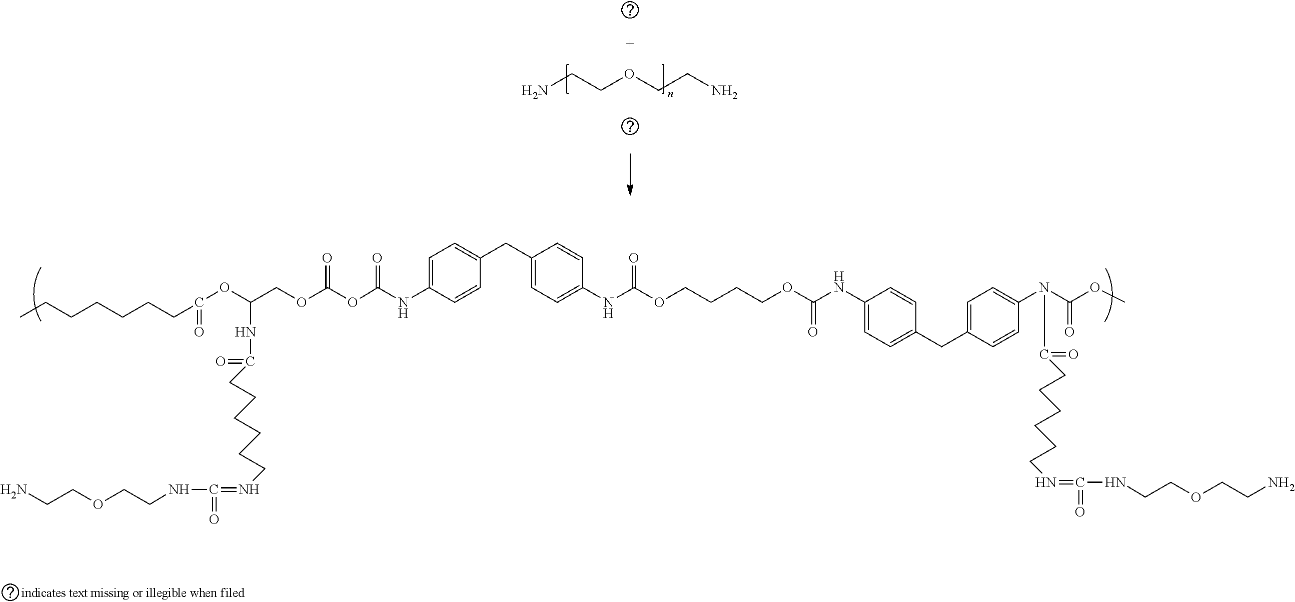

At the third step 207e (depicted below), the APEG conjugated PCU polymer surface is then covalently conjugated to heparin (or any other drug as required) in the presence of 1-ethyl-3-(3-dimethylaminopropyl) carbodiimide (EDAC) and N-hydroxysuccinimide (NHS) to form heparin linked PCU on the polymer's surface.

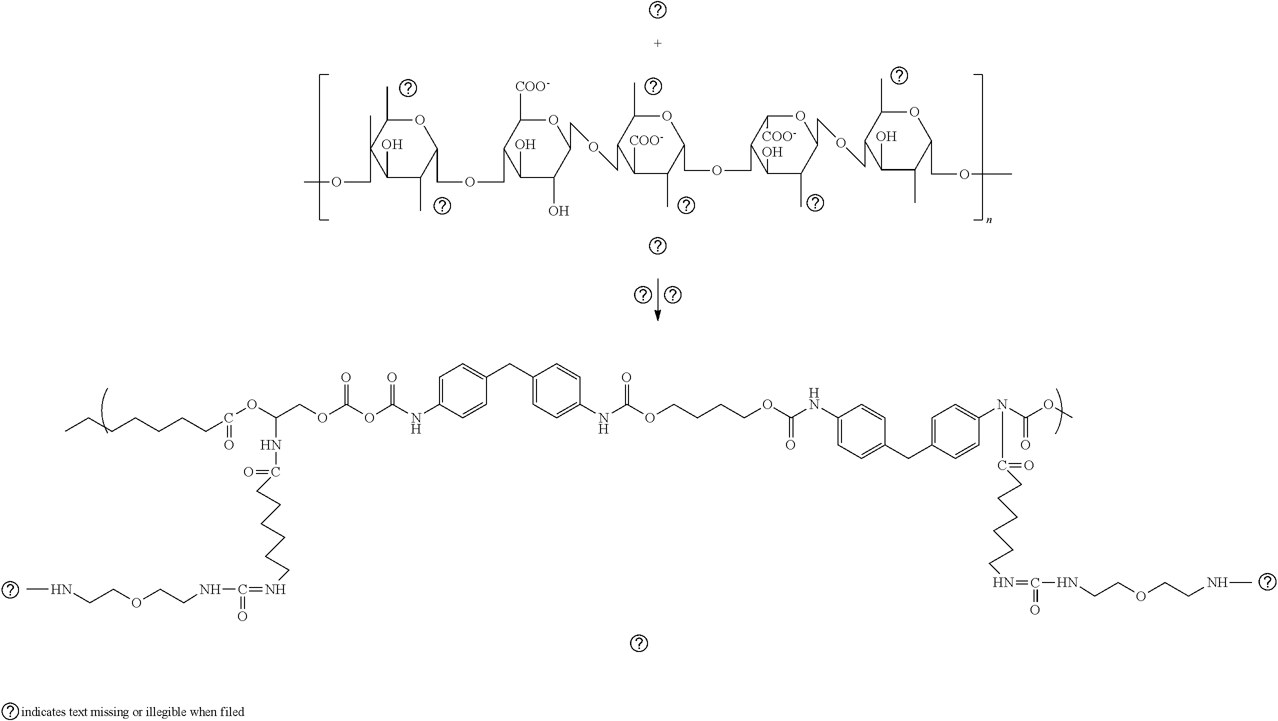

Although, the method 200 as described above includes execution of step 205 before step 207, the method 200 can be performed while executing step 207 before step 205. In other words, the polymeric film may be coated at step 207 before cutting the polymeric film at step 205 to yield the valvular structure 103.

As shown in FIG. 3, the valvular structure 103 includes a top portion 103a and a bottom portion 103b. The top portion 103a and the bottom portion 103b may include a pre-defined shape. In an exemplary embodiment, the top portion 103a may be substantially rectangular (as shown by a dotted line in FIG. 3). The top portion 103a includes an attachment edge 103a1 and a free edge 103a2. In an exemplary embodiment, as shown in FIG. 3, the bottom portion 103b of the valvular structure 103 extends from attachment edge 103a1. The bottom portions 103b may extend equidistant to each other from the attachment edge 103a1 of the top portion 103a.

The free edge 103a2 may be straight or irregular. In an exemplary embodiment, as shown in FIG. 3, the free edge 103a2 includes a zig-zag pattern of alternating dips ‘d’ and peaks ‘p’. The dips ‘d’ may be center aligned with the bottom portion 103b. And, the peaks ‘p’ may be center aligned between two adjacent bottom portion 103b. The aforesaid free edge 103a2 helps in better coaptation of the valvular structure 103.

The top portion 103a between two adjacent bottom portion 103b corresponds to an attachment portion 103a3. The attachment portions 103a3 may be defined as a portion of the valvular structure 103 that is secured to the frame 101 of the THV 100. The attachment portions 103a3 may include a pre-defined shape including but not limited to square, rectangle, etc. In an exemplary embodiment, the attachment portions 103a3 are rectangular shaped. In an exemplary embodiment as shown in FIG. 3, the valvular structure 103 includes two attachment portions 103a3 and two half attachment portions 103a4. In an exemplary embodiment, when the valvular structure 103 is to be secured to the frame 101, the two attachment portions 103a3 are secured to the frame 101, then the two half attachment portions 103a4 are sutured to each other to form a third attachment portion 103a3 and thereafter the third attachment portion 103a3 is secured to the frame 101. In an alternate embodiment, the two half attachment portions 103a4 are first secured together to form the third attachment portion 103a3 (as shown in FIG. 4) and then valvular structure 103 is sutured to the frame 101 with the help of the three attachment portions 103a3. Securement of the attachment portions 103a3 to the frame 101 is described below in detail.

The bottom portion 103b of the valvular structure 103 may each include a bottom edge 103b1 interrupted by attachment portions 103a3 of the top portion 103a. The number of bottom edges 103b1 may depend upon the number of leaflets present in the valvular structure 103. In an exemplary embodiment, the valvular structure 103 includes three U-shaped bottom edges 103b1 as shown in FIG. 3. The bottom edges 103b1 extend away from the attachment edge 103a1 of the top portion 103a of the valvular structure 103. Although the valvular structure 103 of the present invention is explained with U-shaped bottom edges 103b1, other shapes such as scalloped shaped, V-shaped, straight shaped, etc. are within the scope of the teachings of the present invention.

The valvular structure 103 may be mounted within the frame 101 of the prosthetic valve (for example, THV 100) before it is implanted at the target site. It should be noted that though the present invention is explained via examples of transcatheter heart valves, the valvular structure 103 may be mounted within any prosthetic valves.

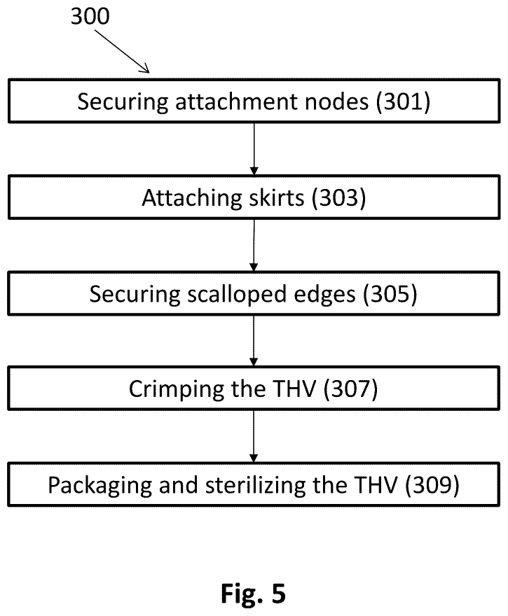

FIG. 5 depicts an exemplary method 300 for assembly of the THV 100 with the valvular structure 103 of the present invention. The method 300 begins at step 301 by attaching the valvular structure 103 to the frame 101 as shown in FIG. 4 to the frame 101. As shown in FIG. 1, the frame 101 may include a plurality of commissure windows 101c disposed at the outflow end 100b of the THV 100. The commissure windows 101c may be disposed equidistant from each other to receive the attachment portions 103a3 of the valvular structure 103. In an exemplary embodiment, each of the attachment portions 103a3 of the valvular structure 103 are sutured to a respective commissure window 101c of the frame 101. The valvular structure 103 is sutured to the frame 101 such that the top portion 103a of the valvular structure 103 is disposed towards the outflow end 100b of the frame 101 and the bottom portion 103b of the valvular structure 103 is disposed towards the inflow end 100a of the frame 101.

In an exemplary embodiment, the two attachment portions 103a3 are secured to the frame 101, then the two half attachment portions 103a4 are sutured to each other to form the third attachment portion 103a3 and thereafter the third attachment portion 103a3 is secured to the frame 101.

In an alternate embodiment, the two half attachment portions 103a4 are first secured together to form the third attachment portion 103a3 and then valvular structure 103 is sutured to the frame 101 with the help of the three attachment portions 103a3.

At next step 303, the inner skirt 102a and the outer skirt 102b may be attached to the frame 101 such that the inner skirt 102a and the outer skirt 102b are disposed towards the inflow end 100a of the frame 101. The inner skirt 102a may be mounted on an inner surface of the frame 101 while the outer skirt 102b is mounted on an outer surface of the frame 101. The inner skirt 102a and the outer skirt 102b may be made up of a predefined material. In an exemplary embodiment, the inner skirt 102a and the outer skirt 102b are made up of the polymer that is used to make the valvular structure 103. The inner skirt 102a and the outer skirt 102b may be made of the same or different polymeric material with respect to each other. The inner skirt 102a and the outer skirt 102b may be made by a method similar to that of the valvular structure 103.

In an alternate embodiment, the inner skirt 102a and the outer skirt 102b are made of polyethylene terephthalate (PET) fabric.

The inner skirt 102a and the outer skirt 102b may be attached to the frame 101 by, without limitation suturing and/or fusion. In an exemplary embodiment, the inner skirt 102a and the outer skirt 102b are attached to the frame 101 by suturing the inner skirt 102a and the outer skirt 102b to the frame 101.

In an alternate embodiment, the inner skirt 102a and the outer skirt 102b are attached to the frame 101 by fusing the inner skirt 102a and the outer skirt 102b to the frame 101 with the help of a medical grade polymeric adhesive. The medical grade polymeric adhesives may include, without limitation acrylic-based adhesives, epoxy resins, polyurethanes, silicone-based adhesives, etc. In an exemplary embodiment, the inner skirt 102a and the outer skirt 102b are attached to the frame 101 by polyurethane. Other functionally equivalent solutions instead of the medical grade adhesives are within the scope of the teachings of the present invention.

At next step 305, the bottom edges 103b1 of the valvular structure 103 are attached to the frame 101. The bottom edge 103b1 may be attached by, without limitation suturing and/or fusion. In an exemplary embodiment, the bottom edge 103b1 of the valvular structure 103 is attached to the frame 101 by suturing the bottom edge 103b1 to the frame 101.

In an alternate embodiment, the bottom edge 103b1 of the valvular structure 103 is attached to the frame 101 by fusing the bottom edge 103b1 of the valvular structure 103 to the frame 101 with the help of a medical grade polymeric adhesive. The medical grade polymeric adhesives may include, without limitation acrylic-based adhesives, epoxy resins, polyurethanes, silicone-based adhesives, etc. In an exemplary embodiment, the bottom edge 103b1 of the valvular structure 103 is attached to the frame 101 by polyurethane, the same polymer that is used for preparation of the valvular structure 103. Other functionally equivalent solutions instead of the medical grade adhesives are within the scope of the teachings of the present invention.

Optionally or alternatively, prior to attaching the bottom edge 103b1 to the frame 101, the bottom edge 103b1 of the bottom portion 103b of the valvular structure 103 may be attached to the inner skirt 102a such that the inner skirt 102a is sandwiched between the bottom edges 103b1 and the frame 101. The valvular structure 103 may be attached to the inner skirt 102a by, without limitation suturing and/or fusion.

In an exemplary embodiment, the bottom edges 103b1 of the valvular structure 103 is attached to the inner skirt 102a by suturing a reinforcing sleeve (not shown). In an exemplary embodiment, the reinforcing sleeve is made of thin PET strips. The reinforcing sleeves enables secure suturing of the valvular structure 103 with the inner skirt 102a and protects the valvular structure 103 from wear and tear.

At step 307, the THV 100 may be crimped on a delivery catheter of a delivery device (not shown). Depending upon the material of the frame 101, the delivery device may be selected. For example, for a self-expandable THV 100, a delivery device for self-expandable implants is selected. Similarly, for a balloon-expandable THV 100, a delivery device having a balloon catheter is selected. Further, in the case of a balloon-expandable THV 100, the THV 100 is not crimped on the delivery catheter. The balloon-expandable THV 100 is only crimped just before the THV 100 is implanted.

At step 309, the THV 100 is packed in a pre-defined packaging and subsequently subjected to sterilization. The packaged THV 100 may be sterilized by, without limitation gas sterilization and/or radiation sterilization. In an exemplary embodiment, the packaged THV 100 is sterilized using Ethylene oxide (EtO) gas. The sterilization of the packaged THV 100 preserves the properties of the polymeric material (such as the valvular structure 103). In an alternate embodiment, the packaged THV 100 is sterilized using gamma beams.

The invention will now be described in more detail by the following non-limiting examples:

### Example 1 (Prior Art): Preparation of a Conventional Polymeric Film for Making a Valvular Structure

A 6% w/v polymer solution was prepared by dissolving 6 grams of polymer (PCU) in anhydrous THF solvent. The polymer solution was continuously stirred by a magnetic stirrer at 1500 rpm to completely dissolve the polymer in the solvent. The polymer solution was then poured inside a petri dish having an area (A) of 254 cm². Thereafter, the said petri dish was kept inside a hot air oven maintained at 60° C. for 6 hours. After said 6 hours, the polymeric film was peeled off from the petri dish using forceps and washed with distilled water. The washed polymeric film was again subjected to high temperature to remove residual moisture.

The resultant polymeric film was found to have bubbles, uneven thickness, haziness and sticky surface.

### Example 2 (Present Invention): Preparation of a Polymeric Film for Making a Valvular Structure

The polymer pellets (PCU) were dried for 24 hours inside a vacuum desiccator to remove moisture. Six grams of polymer was then dissolved in 70 mL DMAc and 30 ml anhydrous THF solvent blend to obtain a 6% w/v polymer solution. The polymer solution was continuously stirred using a magnetic stirrer at 1500 rpm for 8 hours to obtain a homogenous polymer solution. The polymer solution was then filtered through a mesh filter and poured inside a petri dish having an area (A) of 254 cm². Thereafter, the said petri dish was kept inside a vacuum oven where the temperature was gradually increased up to 40° C. and maintained for 3 hours and then again at 70° C. for 8 hours. The said vacuum oven was maintained at 650 mmHg of vacuum pressure. After completion of said 8 hours, the polymeric film was peeled off from the petri dish using forceps washed with distilled water twice. Thereafter, the polymeric film was kept inside the vacuum oven at 50° C. for 1 hour to remove any residual moisture.

The polymer film was found to be clear, without bubbles and homogenous throughout.

### Example 3: Tensile Strength Study

Three samples each of bovine pericardium tissue, conventional polymeric film (as described in example 1) and polymeric film of the present invention (as described in Example 2) were cut in a dog bone shape to test its tensile strength. Each of the dog bone shaped tissue/film was clamped on a universal test machine. The tensile test results, as obtained from bluehill3 software, is tabulated as follows for analysis:

TABLE 2
Tensile Test Data

| | Test | | | | | | | |
|---|---|---|---|---|---|---|---|---|
| | Force @ Peak [N] | | | | Stress @ Peak [MPa] | | | |
| | Sample No. | | | | | | | |
| Sample | 1 | 2 | 3 | Avg. | 1 | 2 | 3 | Avg. |
| Bovine pericardium | 9.72 | 9.06 | 9.20 | 9.32 | 0.97 | 0.91 | 0.92 | 0.93 |
| tissue | | | | | | | | |
| [350 microns thickness] | | | | | | | | |
| Conventional polymeric | 5.82 | 7.43 | 7.87 | 7.04 | 0.66 | 0.72 | 0.79 | 0.72 |
| film | | | | | | | | |
| [180 microns thickness] | | | | | | | | |
| Polymeric film of | 16.21 | 15.89 | 15.92 | 16.00 | 2.02 | 1.91 | 1.96 | 1.96 |
| present invention | | | | | | | | |
| [180 microns thickness] | | | | | | | | |
| Polymeric film of | 7.61 | 9.73 | 9.64 | 8.99 | 0.76 | 0.97 | 0.96 | 0.89 |
| present invention | | | | | | | | |
| [120 microns thickness] | | | | | | | | |
| Polymeric film of | 14.51 | 12.97 | 14.25 | 13.91 | 1.45 | 1.30 | 1.41 | 1.38 |
| present invention | | | | | | | | |
| [150 microns thickness] | | | | | | | | |
| Polymeric film of | 21.36 | 23.64 | 22.74 | 22.58 | 2.86 | 3.18 | 3.05 | 3.03 |
| present invention | | | | | | | | |
| [200 microns thickness] | | | | | | | | |

| | Test | | | | | | | |
|---|---|---|---|---|---|---|---|---|
| | Elongation @ Break [mm] | | | | Youngs Modulus [n/mm²] | | | |
| | Sample No. | | | | | | | |
| Sample | 1 | 2 | 3 | Avg. | 1 | 2 | 3 | Avg. |
| Bovine pericardium | 13.07 | 13.03 | 13.06 | 13.05 | 27.52 | 23.85 | 23.55 | 24.97 |
| tissue | | | | | | | | |
| [350 microns thickness] | | | | | | | | |
| Conventional polymeric | 78.54 | 84.53 | 87.98 | 83.68 | 3.42 | 4.21 | 4.76 | 4.13 |
| film | | | | | | | | |
| [180 microns thickness] | | | | | | | | |
| Polymeric film of | 111.25 | 109.35 | 109.48 | 110.02 | 2.85 | 2.17 | 2.20 | 2.40 |
| present invention | | | | | | | | |
| [180 microns thickness] | | | | | | | | |
| Polymeric film of | 98.57 | 96.58 | 94.99 | 96.71 | 1.62 | 1.43 | 1.30 | 1.45 |
| present invention | | | | | | | | |
| [120 microns thickness] | | | | | | | | |
| Polymeric film of | 103.47 | 105.38 | 103.86 | 104.23 | 2.15 | 1.70 | 1.98 | 1.94 |
| present invention | | | | | | | | |
| [150 microns thickness] | | | | | | | | |
| Polymeric film of | 117.69 | 119.58 | 118.74 | 118.67 | 3.60 | 3.82 | 3.73 | 3.71 |
| present invention | | | | | | | | |
| [200 microns thickness] | | | | | | | | |

Based on the above table, it was concluded that the polymeric film of the present invention has superior mechanical properties compared to the conventional polymeric films. It was further observed that the polymeric film of the present invention, even after having about half the thickness (i.e., 180 microns) of the bovine pericardium tissue, demonstrated superior mechanical properties compared to the bovine pericardium tissue. In fact, even the polymeric film having thickness of only 150 microns outperformed 350 microns thick bovine pericardium tissue. Therefore, the polymeric film of the present invention was considered to be the best candidate to make valvular structures 103 for THVs 100. Not only would the polymeric film of the present invention provide excellent mechanical properties to the valvular structure 103, the polymeric film of the present invention also lowers the overall crimp profile of the THV 100 without compromising its integrity.

### Example 4: In-Vitro Calcification Study

A calcification inducing solution was prepared by adding a calcium compound (for example, CaCl2·2H2O) and a phosphate (K2HPO4) salt in 0.05M tris buffer (pH 7.4). The resultant solution had a calcium to phosphate (Ca/PO4) ratio of about 1.67.

A bovine pericardium tissue (fixed with glutaraldehyde) and the polymeric film of the present invention were cut into small pieces having length 3 cm and width 4 cm. The said pieces of the bovine pericardium tissue and the polymeric film were separately incubated at 37° C. in the above calcification inducing solution for 365 days inside a shaking incubator.

After 365 days, it was observed that the bovine pericardium tissue showed signs of mild calcification. Whereas there was no visible sign of calcification on the polymeric film of the present invention. Thus, it was concluded that the polymeric film of the present invention is extremely resistant to calcification.

The aforesaid conclusion was further verified by placing the THV 100 having a valvular structure 103 made by the polymeric film of the present invention inside an in vitro pulsatile tester machine for accelerated in vitro calcification study using the above calcification inducing solution. The pulsatile machine was operated at 5 Hz frequency. It was reaffirmed that the valvular structure 103 of the THV 100 did not show any sign of calcification and continued to function properly in the accelerated working condition.

### Example 5: Surface Roughness Study

The surface roughness of the polymeric film of the present invention (having thickness 150 micron and 180 micron), conventional polymeric film and bovine pericardium tissue (in duplicate) were measured using Mitutoyo Surftest SJ-410 Surface Roughness Tester Machine and tabulated below for analysis. Generally, electro polished metal tubes such as Nitinol and Cobalt chromium are used for fabrication of cardiovascular stents that possess very smooth surface with no active site for binding of thrombus particles thereby resisting thrombogenesis. Thus, the surface roughness data of electro polished metal tubes were used as a control (reference) for this study.

TABLE 3
Surface Roughness Data

| | Roughness | |
|---|---|---|
| | (Ra) value in | Average Roughness |
| Sample Name | μm | (Ra) value in μm |
| CoCr (cobalt chromium) Tube-1 | 0.139 | 0.130 |
| CoCr (cobalt chromium) Tube-2 | 0.121 | |
| Nitinol Tube-1 | 0.135 | 0.127 |
| Nitinol Tube-2 | 0.119 | |
| Bovine pericardium tissue-1 | 2.596 | 2.735 |
| Bovine pericardium tissue-2 | 2.874 | |
| Conventional polymeric film- | 2.641 | 2.327 |
| 1(with opaque appearance and | | |
| surface stickiness issues) | | |
| Conventional polymeric film- | 2.013 | |
| 2(with opaque appearance and | | |
| surface stickiness issues) | | |
| Conventional polymeric film- | 1.870 | 1.849 |
| 1(with clear appearance having | | |
| bubbles) | | |
| Conventional polymeric film- | 1.829 | |
| 2(with clear appearance having | | |
| bubbles) | | |
| Polymeric Film -1 (150 micron) | 0.142 | 0.201 |
| Polymeric Film -2 (180 micron) | 0.260 | |

It should be noted that high Ra value corresponds to rough surface thereby being more susceptible to thrombogenesis. Thus, it was concluded that not only was the average roughness of the polymeric film of the present invention less than the bovine pericardium tissues, the average roughness of the polymeric film was found to be relatively close to the average roughness of the electro polished metal tubes. Therefore, it was established that similar to electro polished metal tubes, the polymeric film of the present invention provides smooth surfaces which minimizes the propensity for platelet aggregation or any potential thrombus formation and/or pannus formation.

The scope of the invention is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

We claim:

1. A method to prepare a polymeric film for making at least one of a valvular structure (103) and one or more sealing means (102) of a prosthetic heart valve, the method comprising:
pre-processing a polymer for a pre-defined time to reduce residual moisture content of the polymer to less than 0.02% by weight of the polymer;
dissolving the pre-processed polymer in at least one solvent yielding a polymer solution, the polymer solution having a pre-defined concentration, the solvent having water content less than or equal to 0.005% (w/v);
pouring the polymer solution into an enclosed surface having a pre-defined area;
subjecting the polymer solution to a drying technique at a first pre-defined temperature(s) and a first pre-defined pressure(s) for a first pre-defined time period(s) to yield a polymeric film, the polymeric film having a thickness ranging from 150 microns to 220 microns;
washing the polymeric film; and
subjecting the polymeric film to the drying technique at a second pre-defined temperature(s) for a second pre-defined time period(s).

2. The method as claimed in claim 1, wherein the step pre-processing the polymer for the pre-defined time includes
selecting the polymer from a group of polyurethane or block co-polymers thereof including polyurethane, poly ether urethane, and polycarbonate urethane; and
drying the polymer in one of a vacuum desiccator, a vacuum oven, a desiccant type de-humidifying dryer, or a hot air oven for 12 hours to 24 hours.

3. The method as claimed in claim 1, wherein the step of dissolving the polymer in at least one solvent includes dissolving the polymer in at least one solvent at a concentration ranging from 3% to 11% (w/v).

4. The method as claimed in claim 1, wherein the step of dissolving the polymer in at least one solvent includes dissolving the polymer in at least one of dimethyl acetamide (DMAc), tetrahydrofuran (THF), or dimethyl sulfoxide (DMSO).

5. The method as claimed in claim 1, wherein the step of dissolving the polymer in at least one solvent includes dissolving the polymer in a blend of two solvents blended in a predefined ratio of 90:10, 80:20, 70:30, 60:40, 50:50, 40:60, 30:70, 20:80, or 10:90.

6. The method as claimed in claim 1, wherein the step of pouring the polymer solution includes pouring the polymer solution within the enclosed surface having the pre-defined area ranging from 80 cm² to 315 cm².

7. The method as claimed in claim 1, wherein the step of subjecting the polymer solution to a drying technique at the first pre-defined temperature(s) and the first pre-defined pressure(s) for the first pre-defined time period(s) includes maintaining the polymer solution at the first pre-defined temperature(s) ranging from 40° C. to 80° C. and the first pre-defined pressure(s) ranging from 600 mmHg to 700 mmHg for a first pre-defined time period(s) ranging from 5 hours to 10 hours.

8. The method as claimed in claim 1, wherein the step of subjecting the polymeric film to the drying technique at the second pre-defined temperature(s) for the second pre-defined time period(s) includes maintaining the polymeric film at 37° C. for 24 hours under atmospheric pressure.

9. The method as claimed in claim 1, wherein the step of subjecting the polymeric film to the drying technique at the second pre-defined temperature(s) for the second pre-defined time period(s) includes maintaining the polymeric film at 60° C. for 3-4 hours.

10. The method as claimed in claim 1, wherein after preparing the polymeric film, the method includes coating the polymeric film with a pre-defined coating.

11. The method as claimed in claim 1, wherein after preparing the polymeric film, the method includes cutting the polymeric film to a pre-defined shape via one of laser cutting, water knife, or ultrasonic trimming to form the valvular structure (103).

12. A prosthetic heart valve, comprising:
a frame (101);
one or more sealing means (102) attached to the frame (101); and
a polymeric film cut to a predefined shape to yield a valvular structure (103), the valvular structure (103) attached to the frame (101);
wherein, the polymeric film is made from a polymer solution, the polymer solution including a polymer dissolved in at least one solvent, the polymer solution subjected to a drying technique at a first pre-defined temperature(s) and a first pre-defined pressure(s) for a first pre-defined time period(s);
wherein the polymer is dehydrated;
wherein, the at least one solvent is anhydrous;
wherein the polymeric film subjected to a drying technique at a second pre-defined temperature(s) for a second pre-defined time period(s).

13. The prosthetic heart valve as claimed in claim 12, wherein the valvular structure includes three discrete leaflets.

14. The prosthetic heart valve as claimed in claim 12, wherein the valvular structure (103) includes a single continuous structure having a thickness ranging from 150 microns to 220 microns.

15. A prosthetic heart valve, comprising:
a frame (101);
a valvular structure (103) supported within the frame (101); and
one or more sealing means (102) attached to the frame (101); at least one of the one or more sealing means (102) made from a polymeric film;
wherein, the polymeric film is made from a polymer solution, the polymer solution including a polymer dissolved in at least one solvent, the polymer solution subjected to a drying technique at a first pre-defined temperature(s) and a first pre-defined pressure(s) for a first pre-defined time period(s);
wherein the polymer is dehydrated;
wherein, the at least one solvent is anhydrous;
wherein the polymeric film subjected to a drying technique at a second pre-defined temperature(s) for a second pre-defined time period(s).
